(19) United States
(12) Patent Application Publication
(10) Pub. No.: US 2012/0016039 A1
(43) Pub. Date: Jan. 19, 2012

(54) USE OF OLIVE-OIL-DERIVED COMPOUNDS AS SURFACTANTS

(75) Inventors: Sergio Amari, Lecco (IT); Alain Thibodeau, De-Desmaures (CA)

(73) Assignee: B & T S.R.L., Arcore, Milano (IT)

(21) Appl. No.: 13/140,567

(22) PCT Filed: Dec. 24, 2008
(86) PCT No.: PCT/IT08/00798

§ 371 Date: Oct. 5, 2011

(51) Int. Cl. A61K 8/37

(52) U.S. Cl. 514/785

(57) ABSTRACT

The use of—olive-oil—derived compounds as surfactants is described, wherein such olive-oil-derived compounds are obtained by olive oil transesterification. Preferably, such compounds are a mixture-composed of an emollient and of anionic surfactants. As an emollient a product obtained via transesterification of olive oil with glycerine derivatives is preferably used most preferably olive oil PEG-7 esters, whereas the anionic surfactant is obtained from the emollient by causing it to react with monochloroacetic acid most preferably Na PEG-7 olive oil Carboxylate.

### FIELD OF THE INVENTION

This invention refers to the use of olive-oil-derived compounds as cosmetic surfactants, which exhibit features very similar to human skin.

### BACKGROUND OF THE INVENTION

As is well-known, human skin is coated by a lipidic layer which is produced on the surface of the epidermis and combines with water molecules. More precisely, such lipidic layer is in fact a hydrolipidic layer which, through a skin barrier function, protects the skin from the action of harmful agents, which can affect and even damage the skin. Damages to the skin can be cosmetic damages, which have mainly an aesthetic effect, as well as dermatological ones, which can have harmful effects on health. The hydrolipidic layer embeds keratinocytes and corneocytes. Moreover, the hydrolipidic layer provides a high surface energy, giving the skin a hydrophilic character, making the skin surface wettable.

Lipids of the hydrolipidic layer also diffuse inwards within the lipid matrix of the stratum corneum. In this process, lipids in the hydrolipidic layer merge with the lipid matrix already established between the keratinocytes of the stratum corneum. The hydrolipidic layer is thus important not only for the skin's sensory aspect and wettability, but it also functionally contributes to the skin's barrier function.

The hydrolipidic layer, despite its very effective protection, can be removed, however, in a relatively easy fashion and there are a number of quite dangerous agents in this respect. One of the most common of such agents is represented by common soaps and detergents. It must be pointed out that the hydrolipidic layer normally has a slightly acidic pH. Most soaps and detergents are basic, so that a part of them reacts with the layer and removes it. In this way, delipidised areas occur on the surface of the epidermis. Surfactants contained in soaps interact with proteins causing denaturation thereof, which triggers skin irritation. This is why the repeated use of soaps and detergents often results in skin irritation and rash (erythema).

Furthermore, surfactants and detergents can destroy the integrity of the hydrolipidic layer, thereby causing lipid-depleted areas on the surface of the epidermis. This delipidifying action leads to a change in skin topography and may cause alterations of the skin's sensory function. Results could be skin dryness, roughness, desquamation and a taut feeling. Moreover, surfactant micelles and surfactant monomers can also penetrate into the upper layers of the epidermis, interacting with stratum corneum lipids and proteins, respectively. Ultimately, soap applications might lead to a deterioration of the barrier function and to skin erythema.

Furthermore, the removal of the hydrolipidic layer results in weakening of the skin, which can therefore be attacked by bacteria, viruses and fungi with further possible diseases (e.g. verrucae).

Also the repeated contact with water, especially with humidity, can lead to the partial removal of the hydrolipidic layer and proteins can be denaturated by cold. This is the reason why skin is often irritated in winter, especially in humid places.

A first attempt to solve the problem has been the use of acidic, usually rather expensive soaps, which can alleviate the above problems. However, they completely fail to protect the skin and their action is limited to slowing down the removal of the hydrolipidic layer and to killing a part of fungi, viruses and bacteria; in any case, surfactants still act as skin irritants.

The object of this invention is to provide a soap or detergent which is as effective as most soaps and detergents and which does not damage the skin, especially suitable for people who need to wash their hands very often.

### SUMMARY OF THE INVENTION

The above object is achieved by this invention, referring to the use of olive-oil-derived compounds as surfactants, characterised in that such olive-oil-derived compounds are obtained by olive oil transesterification.

### BEST WAY TO CARRY OUT THE INVENTION

The present invention refers to the use of compounds derived from the transesterification of olive oil as surfactants. Olive oil is a mixture, the composition of which depends greatly on the particular olives from which it is obtained, on the particular year, on the preparation procedure and on other factors. In any case, olive oil normally contains a well-defined proportion of components (palmitic acid 7.0-20.0, palmitoleic acid ≦3.5, stearic acid ≦5.0, eicosenoic acid 0.4, oleic acid 56.0-85.0, linoleic acid 4.0-20.0, arachidic acid ≦0.6 and linolenic acid ≦0.1, all figures being % by weight), so that it can be considered a well-defined composition. Most olive oil components are fatty acid esters, particularly unsaturated fatty acid esters.

The transesterification reaction is a well-known chemical reaction, transforming an ester into another ester by reaction with an alcohol. Transesterification is reported in the main books of organic chemistry and it is not further explained, since a skilled person is usually able to perform it.

The effect of a mild surfactant can be obtained in a particularly effective way by mixing an emollient and anionic surfactants, both obtained by olive oil transesterification. As an emollient, a product obtained via transesterification of olive oil with glycerine derivatives is particularly suitable. Better results are obtained if the glycerine derivative is a pre-ethoxylated glycerine, particularly if the pre-ethoxylation relates to 7.5 moles of the glycerine. A particularly good emollient is olive oil transesterified with 7.5 moles of pre-ethoxylated glycerine, known under the INCI name of olive oil PEG-7 esters. Such a product can be considered as a polymer having its acid chains bonded to n moles of polyethylene glycol, wherein n is an integer from 2 to 7. This emollient has a high fat content (more than 35%), in spite of its high water-solubility. This degree of water solubility is uncommon in this class of compounds and can usually be obtained only by directly ethoxylating the fatty acid. This direct ethoxylation results in a reduced fat content, which drops to below 10%. The fact that fats are at least below 35% in this emollient makes it very useful against possible skin irritation. A particularly suitable anionic surfactant is the one obtained from the emollient, by causing it to react with monochloroacetic acid, especially the one having the INCI name Sodium PEG-7 Olive Oil Carboxylate. The introduction of a carboxy group increases foam build-up, so that a surfactant effect is achieved without lowering the emollient power. This fact is useful in creating a mild surfactant which does not lead to a damage of the hydrolipidic layer of the skin. Indeed, these products exhibit a very high molecular similarity to the composition of the hydrolipidic film which coats the skin surface. Therefore, it is assumed that they reduce skin damage in that they prevent the deterioration of the hydrolipidic film by replenishing it.

### Experimental Section

The action of the claimed products was clinically tested. Soaps were formulated in the presence or in the absence of a mixture containing olive oil PEG-7 esters (component A) and Sodium PEG-7 Olive Oil Carboxylate (component B). The ingredients were individually tested. The effect of three soaps (alone, with component A and with component B) was verified on the skin, by testing trans-epidermal water loss (TEWL), skin hydration (corneometry) and skin irritation (colorimetry). Soaps containing either component A or component B exhibited a decrease in TEWL, reduced loss of skin hydration and reduced skin irritation.

As a surfactant, Sodium PEG-7 Olive Oil Carboxylate (in the following Olivem 400) was tested through an in vitro alternative to measure potential eye irritation. This assay quantifies the effects of surfactants on the lysis of erythrocytes (H) and on the three-dimensional conformational changes of proteins (D). Briefly, when erythrocytes are incubated in the presence of surfactants, the latter may interact with the membrane lipids of erythrocytes and cause lysis thereof, thereby releasing intracellular proteins (mostly hemoglobin). Hemoglobin release can be quantified by optical density measurements and is calculated as parameter H. In a second phase, surfactants may interact with proteins causing denaturation thereof. This will lead to a three-dimensional conformational change (denaturation) of proteins that can also be quantified by optical density measurements. Protein denaturation is calculated as parameter D. Results are expressed as H/D ratio by weight. Products having an H/D ratio of 10 or above are categorised as non pro-irritant, while those showing an H/D ratio below 10 are categorised as potentially pro-irritant. It is noteworthy that most surfactants become pro-irritant when they interact with proteins of the stratum corneum, causing protein denaturation. An H/D ratio as high as possible is therefore desirable. A comparison of the H/D ratio of Olivem 400 with standard surfactants showed that Olivem 400 exhibits a higher H/D ratio by 2.19-89.28 times.

Olivem 400 can also reduce the aggressiveness of other surfactants when mixed with them. A solution of 17.1% sodium lauryl ether sulphate was incubated in the presence of 7.2% of sodium cocoamphoacetate (NCA), decyl glucoside (DG) or Olivem 400. All the above-cited agents are mild surfactants. The admixtures with Olivem 400 led to an H/D higher by 1.12-2.01 times than with the others, the H/D value being 12.1. Therefore the products according to this invention can be used both alone (as surfactants) and in a mixture with other surfactants (as agents making the other surfactants milder). Although a surfactant produced by inserting the products of this invention into a conventional surfactant is stronger (i.e. less mild) than the products of this invention alone, it is nevertheless useful to make the conventional surfactants less harmful.

The ability of oil PEG-7 esters (in the following Olivem 300) and Olivem 400 to make soaps more compatible with skin physiology was clinically tested. A base bar formulation was developed and used either alone (control) or additioned with 2% Olivem 300 or 2% Olivem 400. The three soap bars to be tested were diluted at 10% in water and the solution was applied as a semi-occlusive patch to the skin of healthy subjects for 24 hours. The use of a semi-occlusive patch is important to allow for water evaporation of the soap solution.

After 24 hours, the patch was removed and three parameters were clinically evaluated: TEWL (trans epidermal water loss), skin hydration and skin erythema at various times after having removed the patch. Immediately after patch removal, contact with the base soap bar caused an excessive increase in TEWL. With time, the skin barrier function reorganises itself as the TEWL value decreases. Even 60 minutes after having removed the patch, the TEWL value is above the baseline, showing that the skin barrier function has not completely recovered yet. The addition of Olivem 300 or 400 minimises the increase of TEWL by 61.1% (Olivem 300) or by 47.8% (Olivem 400).

Results obtained through corneometry measurements show that the loss of skin hydration is prevented by the admixture according to this invention.

This invention provides an ideal new concept for developing dermatologically-compatible detergent formulations. This is due to the similarity that the used compounds according to this invention display, when compared to skin composition.

1.-9. (canceled)

10. A surfactant, comprising at least one olive-oil-derived compound obtained by transesterification of olive oil.

11. The surfactant according to claim 10, wherein the at least one compound obtained by transesterification of olive oil comprises a mixture of an emollient obtained by transesterification of olive oil and anionic surfactants obtained by transesterification of olive oil.

12. The surfactant according to claim 11, wherein the emollient is a transesterification of product of olive oil and one or more glycerine derivatives.

13. The surfactant according to claim 12, wherein the glycerine derivative comprises a pre-ethoxylated glycerine.

14. The surfactant according to claim 13, wherein the emollient is an olive oil transesterified with 7.5 moles of pre-ethoxylated glycerine.

15. The surfactant according to claim 14, wherein the emollient is the olive oil transesterified with 7.5 moles of pre-ethoxylated glycerine known under the INCI name of olive oil PEG-7 esters.

16. The surfactant according to claim 11, wherein the anionic surfactant is obtained by reacting the emollient with monochloroacetic acid.

17. The surfactant according to claim 12, wherein the anionic surfactant is obtained by reacting the emollient with monochloroacetic acid.

18. The surfactant according to claim 13, wherein the anionic surfactant is obtained by reacting the emollient with monochloroacetic acid.

19. The surfactant according to claim 14, wherein the anionic surfactant is obtained by reacting the emollient with monochloroacetic acid.

20. The surfactant according to claim 15, wherein the anionic surfactant is obtained by reacting the emollient with monochloroacetic acid.

21. The surfactant according to claim 15, wherein the anionic surfactant is the compound having the INCI name Sodium PEG-7 Olive Oil Carboxylate.

22. The surfactant according to claim 10, further comprising one or more other surfactants.

23. The surfactant according to claim 11, further comprising one or more other surfactants.

24. The surfactant according to claim 12, further comprising one or more other surfactants.

25. The surfactant according to claim 13, further comprising one or more other surfactants.

26. The surfactant according to claim 14, further comprising one or more other surfactants.

27. The surfactant according to claim 15, further comprising one or more other surfactants.

28. The surfactant according to claim 16, further comprising one or more other surfactants.

29. The surfactant according to claim 21, further comprising one or more other surfactants.
